(19) United States
(12) Patent Application Publication
(10) Pub. No.: US 2011/0033527 A1
(43) Pub. Date: Feb. 10, 2011

(54) Opthalmic compositions of cyclosporin

(76) Inventors: Nian Wu, North Brunswick, NJ (US); Brian Charles Keller, Antioch, CA (US)

Correspondence Address: Lee Pederson, 712 East Main Street, Sleepy Eye, MN, 56085, US

(21) Appl. No.: 12/802,200

(22) Filed: Jun. 1, 2010

Related U.S. Application Data
(60) Provisional application No. 61/217,627, filed on Jun. 2, 2009.
(60) Provisional application No. 61/273,656, filed on Aug. 5, 2009.

(51) Int. Cl. A61K 9/48; A61K 38/13; A61P 27/02; A61P 29/00; A61P 37/06

(52) U.S. Cl. 424/451; 514/20.5

(57) ABSTRACT

Lipid-polymer compounds are used to solubilize cyclosporin. Diacylglycerol-polyethyleneglycols (DAG-PEGs) are especially useful in this regard. A preferred embodiment of the invention is an aqueous solution of cyclosporin suitable for opthalmic use.

### RELATED APPLICATIONS

This application claims priority based upon U.S. provisional patent application No. 61/217,627 entitled “Pure PEG-Lipid Conjugates” and filed Jun. 2, 2009; and based upon U.S. provisional patent application No. 61/273,656 entitled “Opthalmic Compositions of Cyclosporin” and filed August 5, 2009.

### FIELD OF THE INVENTION

The present invention relates to cyclosporin formulations and to processes for preparing these compositions. More particularly, the present invention relates to employing lipid-polymer conjugates to initially solubilize cyclosporin.

### BRIEF DESCRIPTION OF THE INVENTION

Lipid-polymer compounds are used to solubilize cyclosporin. Diacylglycerol-polyethyleneglycols (DAG-PEGs) are especially useful in this regard. A preferred embodiment of the invention is an aqueous solution of cyclosporin suitable for opthalmic use.

### BACKGROUND OF THE INVENTION

The cyclosporins are cyclic oligopeptides of microbiological origin, a class of structurally distinctive, cyclic, poly-N-Methylated undecapeptides, possessing common pharmacological, particularly immunosuppressive, anti-inflammatory and/or anti-parasitic activity. Due to its immunosuppressive effect, cyclosporin is widely used: in kidney, liver, heart, lung, pancreas, skin and cornea transplantations in order to prevent the rejection of the transplanted organ; in bone marrow transplantations to inhibit the antibody production of the transplanted bone marrow against the host organism; further for healing autoimmune diseases such as rheumatoid arthritis, diabetes mellitus I, systematic lupus erythematosis, scleroderma, Wegener's granulomatosis, eosinophilic fascitis, primary liver cyrrhosis, Graves' and Crohn's diseases. Similarly, it is used for the treatment of myasthenia gravis, multiplex sclerosis and psoriasis. The first of the cyclosporins isolated was the naturally occurring fungal metabolite cyclosporin, also known as cyclosporin A or cyclosporine.

Cyclosporin exhibits very poor solubility in water and, as a consequence, suspension and emulsion forms of the drug have been developed for oral administration and for injection. A topical emulsion of cyclosporin for treating keratoconjunctivitis sicca has been marketed since 2002. Cyclosporin is also available as a preparation for the treatment of atopic dermatitis in dogs. Unlike the human form of the drug, the lower doses used in dogs indicates that the drug acts as an immuno-modulator and has fewer side-effects than in man. The benefits of using this product for dogs includes the reduced need for concurrent therapies to bring the condition under control.

A disadvantage with most of cyclosporin compositions lies in that vegetable oils are used as carrier additives, which endows an unpleasant oily taste. Also, these compositions disintegrate during storage whereby a further undesired alteration may occur in the taste and odor of the compositions. Although the rancidification may be limited by addition of antioxidants, this process cannot completely be eliminated. Thus, the oral compositions prepared according to present methods can be commercialized with only a relatively short shelf-life.

### DETAILED DESCRIPTION OF THE INVENTION

Embodiments of the present invention are described herein in the context of compositions and methods for formulating cyclosporin. Those of ordinary skill in the art will realize that the following detailed description of the present invention is illustrative only and is not intended to be in any way limiting. Other embodiments of the present invention will readily suggest themselves to such skilled persons having the benefit of this disclosure.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Those of ordinary skill in the art will realize that the following description of the present invention is illustrative only and not in any way limiting. Other embodiments of the invention will readily suggest themselves to such skilled persons having the benefit of this disclosure.

The present invention provides a class of new cyclosporine formulations that are suitable for therapeutic use as ophthalmic, oral, topical and intervenous administration of cyclosporin. These formulations are both chemically and microbiologically stable as well as offering improved pharmacokinetic profiles.

Due to the greater solubility of cyclosporin in the present PEG-lipids, it is convenient and flexible to formulate the drugs in various dosage forms, for example eye drops for ophthalmic dosage.

The present invention also discloses a class of pharmaceutically acceptable PEG-conjugates to be used as a drug delivery vehicle in association with cyclosporin, said pharmaceutically acceptable carriers including: pegylated mono- or di-fatty acid esters of glycerol.

One aspect of the present invention relates to a pharmaceutical acceptable PEG-lipid carrier system in association with cyclosporin in pharmaceutical formulations. The pharmaceutical compositions of the present invention may be administered orally by capsule or liquid; or in liquid form for parenteral, intramuscular or intravenous administration. In a preferred embodiment, the invention provides a composition in a form appropriate or adapted for oral administration, in particular in the form of capsules, drink solutions or dry powder for reconstituting; or a Soxhlet form prepared by standard techniques known in the art, such as by spray coating on deposition. It is also a preferred embodiment that the PEG-lipid containing formulation to be used as an ophthalmic preparation, i.e., eye drops. It is further preferred that the PEG-lipid containing formulation to be used as a topical application for re-growing hair.

Cyclosporin, which is used as the pharmaceutically active ingredient in the composition according to the present invention, is a cyclic peptide compound having useful immunosuppressive activity and anti-inflammatory activity. Although various cyclosporins, such as cyclosporin A and the like can all be used as the cyclosporin component in the present invention, cyclosporin A is preferred.

The invention relies on employing one or more amphipathic PEG-lipid conjugates to solubilize cyclosporin. Such conjugate or conjugates may be capable of spontaneously forming liposomes in aqueous solution, as taught in U.S. Pat. No. 6,610,322, which is hereby incorporated by reference. Diacylglycerol-polyethyleneglycols (DAG-PEGs) are preferred solubilizing agents. The hydrophobic lipid portion of the molecules interacts with the cyclosporin while the polymer chain confers solubility in aqueous solution. Since the acyl groups and the PEG chains are responsible for the contributing the properties that allow solubilization, the specific chemical bonds to the glycerol backbone may be varied within the scope of the invention and within the meaning of diacylglycerol-polyethyleneglycol. The relative positions o the lipids and PEG chain on the backbone are not crucial. Also, since the lipid groups and the PEG chains are responsible for the contributing the properties that allow solubilization, other similar molecules may be employed. Such molecules include monoacylglycerol-dipolyethylene glycols, molecules where sterols are substituted for acyl groups, and molecules having alternative backbones to glycerol.

Cyclosporin may be associated with a carrier system comprising at least one of the PEG-lipids listed in Tables 1 and 2. In aqueous solution, the PEG-lipids are present in a total lipid concentration range from 0.5% to 20%, which is compatible achieving maximum cyclosporin solubility.

TABLE 1
PEG-lipids for use in the present invention

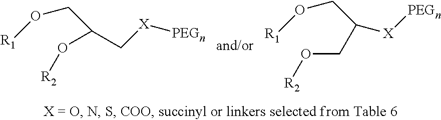

| Symbol | R₁ | R₂ | n (PEG) |
|---|---|---|---|
| GDO-PEGₙ | Oleoyl | Oleoyl | 4 to 12 |
| GDM-PEGₙ | Mystroyl | Mystroyl | 4 to 12 |
| GDS-PEGₙ | Stearyl | Stearyl | 4 to 23 |
| GDP-PEGₙ | Palmitoyl | Palmitoyl | 4 to 23 |
| GDC-PEGₙ | Cholyl | Cholyl | 4 to 23 |

TABLE 2
PEG-lipids for use in the present invention

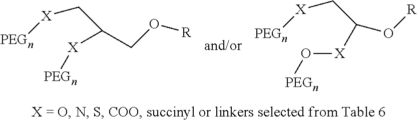

| Symbol | R | n (PEG) |
|---|---|---|
| GMO-diPEGₙ | Oleoyl | 4 to 23 |
| GMM-diPEGₙ | Mystroyl | 4 to 23 |
| GMS-diPEGₙ | Stearyl | 4 to 23 |
| GMP-diPEGₙ | Palmitoyl | 4 to 23 |
| GMC-diPEGₙ | Cholyl | 4 to 23 |

Other possible lipids (R groups) are summarize in Tables 3, 4 and 5.

Table 3
Saturated lipids for use in the invention:

| common | | | | Melting |
|---|---|---|---|---|
| name | IUPAC name | Chemical structure | Abbr. | point (° C.) |
| Caprylic | Octanoic acid | CH₃(CH₂)₆COOH | C8:0 | 16-17 |
| Capric | Decanoic acid | CH₃(CH₂)₈COOH | C10:0 | 31 |
| Lauric | Dodecanoic acid | CH₃(CH₂)₁₀COOH | C12:0 | 44-46 |
| Myristic | Tetradecanoic acid | CH₃(CH₂)₁₂COOH | C14:0 | 58.8 |
| Palmitic | Hexadecanoic acid | CH₃(CH₂)₁₄COOH | C16:0 | 63-64 |
| Stearic | Octadecanoic acid | CH₃(CH₂)₁₆COOH | C18:0 | 69.9 |
| Arachidic | Eicosanoic acid | CH₃(CH₂)₁₈COOH | C20:0 | 75.5 |
| Behenic | Docosanoic acid | CH₃(CH₂)₂₀COOH | C22:0 | 74-78 |

TABLE 4
Unsaturated lipids for use in the invention

| | | Δ^{x} | |
|---|---|---|---|
| | | Location of | # carbon/ |
| Name | Chemical structure | double bond | double bonds |
| Myristoleic acid | CH₃(CH₂)₃CH═CH(CH₂)₇COOH | cis-Δ⁹ | 14:1 |
| Palmitoleic acid | CH₃(CH₂)₅CH═CH(CH₂)₇COOH | cis-Δ⁹ | 16:1 |
| Oleic acid | CH₃(CH₂)₇CH═CH(CH₂)₇COOH | cis-Δ⁹ | 18:1 |
| Linoleiacid | CH₃(CH₂)₄CH═CHCH₂CH═CH(CH₂)₇COOH | cis,cis-Δ⁹,Δ¹² | 18:2 |
| α-Linolenic acid | CH₃CH₂CH═CHCH₂CH═CHCH₂CH═CH(CH₂)₇COOH | cis,cis,cis-Δ⁹,Δ¹²,Δ¹⁵ | 18:3 |
| Arachidonic acid | CH₃(CH₂)₄CH═CHCH₂CH═CHCH₂CH═CHCH₂CH═CH(CH₂)₃COOH | cis,cis,cis,cis-Δ⁵Δ⁸,Δ¹¹,Δ¹⁴ | 20:4 |
| Erucic acid | CH₃(CH₂)₇CH═CH(CH₂)₁₁COOH | Cis-Δ¹³ | 22:1 |

TABLE 5
Steroid acid and analogues for use in the invention

| Name | Chemical Structure | Other Name |
|---|---|---|
| Cholic acid | 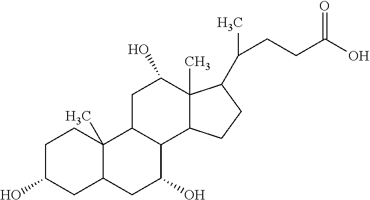 | 3α,7α,12α-trihydroxy- 5β-cholanoic acid |
| Desoxycholic acid | 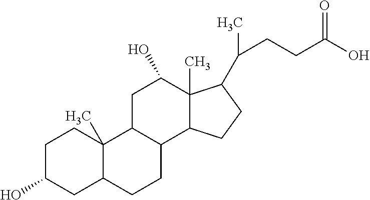 | 3α,12α-Dihydroxy-5β- cholanic acid |
| 5-Cholenic acid-3β-ol | 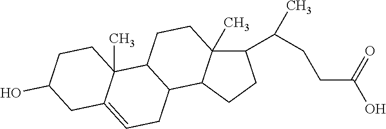 | 3β-Hydroxy-5-cholen- 24-oic acid |
| Dehydrocholic acid | 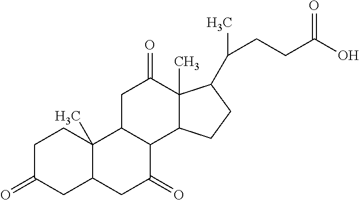 | 3,7,12-Trioxo-5β- cholanic acid |
| Glycocholic acid | 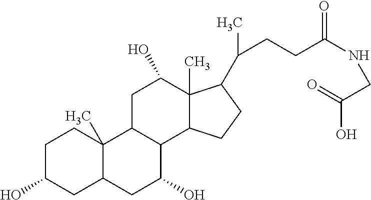 | N-(3α,7α,12α- Trihydroxy-24- oxocholan-24-yl)- glycine |
| Glycodeoxycholic acid | 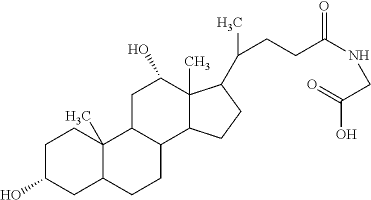 | N-(3α,12α-Dihydroxy- 24-oxocholan-24- yl)glycine |
| Chenodeoxycholic acid | 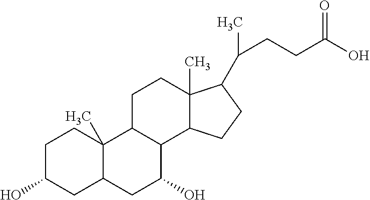 | 3α,7α-dihydroxy-5β- cholanic acid |
| Glycochenodeoxycholic acid | 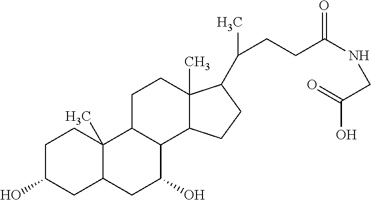 | N-(3α,7α-Dihydroxy- 24-oxocholan-24- yl)glycine |
| Ursodeoxycholic acid | 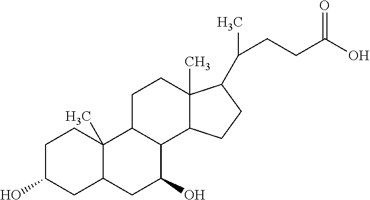 | Ursodiol |
| Lithocholic acid | 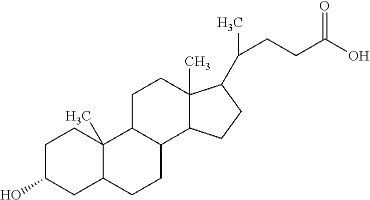 | 3α-Hydroxy-5β-cholan- 24-oic acid |
| Hyodeoxycholic acid | 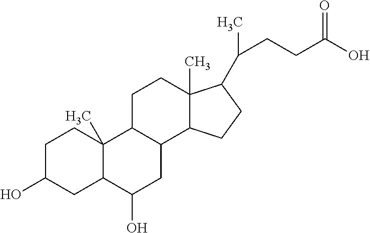 | 3α,6α-Dihydroxy-5β- cholan-24-oic acid |
| 5β-Cholanic acid-3,7- dione | 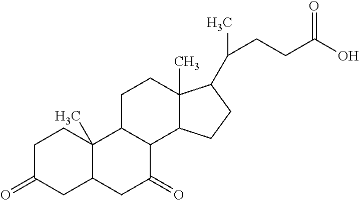 | 3,7-Diketo-5β-cholan- 24-oic acid |

Some additional linkers for attaching the PEG to the backbone are summarized in Table 6.

TABLE 6
Linkers for use in the invention

| No | Symbol | Linker |
|---|---|---|
| 1 | N₁ | 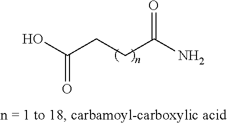 |
| 2 | N₂ | 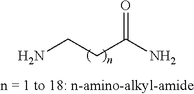 |
| 3 | N₃ | 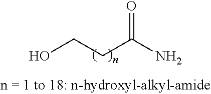 |
| 7 | N₇ | 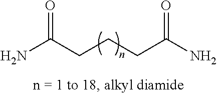 |
| 8 | N₈ | 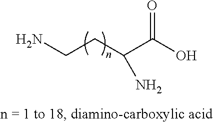 |
| 9 | N₉ | 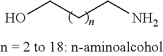 |
| 10 | N₁₀ | 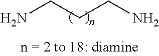 |
| 11 | N₁₁ | 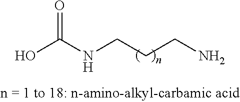 |
| 12 | N₁₂ | 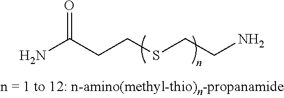 |
| 13 | S₁ | 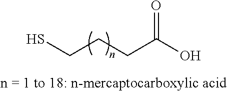 |
| 14 | S₂ | 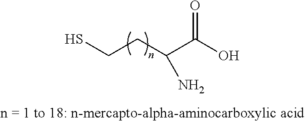 |
| 15 | S₃ | 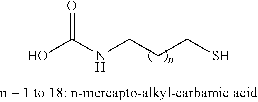 |
| 16 | S₄ | 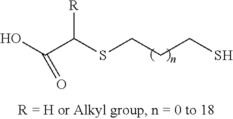 |
| 17 | S₅ | 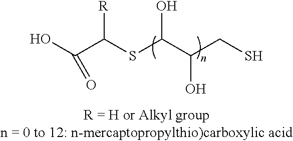 |
| 18 | S₆ | 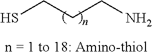 |
| 19 | S₇ | 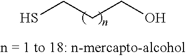 |
| 20 | S₈ | 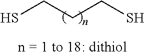 |
| 21 | S₉ | 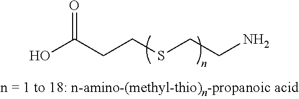 |
| 22 | Ac₁ | 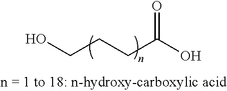 |
| 23 | Ac₂ | 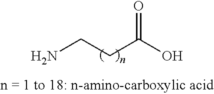 |
| 24 | Ac₃ | 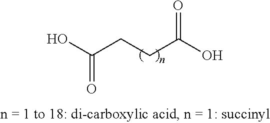 |
| 25 | Ac₄ | 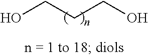 |
| 26 | Ac₅ | 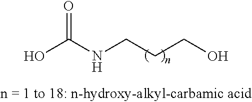 |
| 27 | Ac₆ | 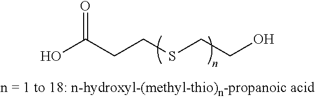 |

The methods and compositions of the invention may include the addition of excipients not described herein without departing from the invention.

The present invention involves solubilizing cyclosporine by using one or more amphipathic PEG conjugates. Diacylglycerol-polyethyleneglycols (DAG-PEGS) are preferred conjugates, in which acyl chains comprise the lipophilic portion of the conjugate. Other suitable amphipathic conjugates include monoacyl PEGs and PEG-steroid conjugates.

The critical step for solubilization is combining cyclosporine with an amphipathic PEG conjugate which is liquid at the temperature of solubilization. For formulating at room temperature, this means employing a conjugate having a melting temperature less than about 25 degrees Centigrade. By performing solubilization at elevated temperatures, conjugates with higher melting temperatures may be used. Such solubilization can be done by adding the cyclosporine to the conjugate only, or by adding the cyclosporine to the conjugate in aqueous solution.

For applications where a liquid form is desired (e.g., eye drops, oral solution, IV solution, or topical solution), such solubilization essentially results in a usable formulation of the drug. For applications where a solid or semi-solid form is more desirable (e.g., oral capsule or topical cream), a second amphipathic PEG conjugate having a higher melting temperature is added after the initial solubilization.

Preferred formulations of cyclosporine according to the present invention include:

Eye drops: preferable concentration of cyclosporine is 0.01 to 1% by weight, more preferable is 0.05 to 0.05%, most preferable is 0.05 to 0.1%. The preferable ratio of conjugate to the drug (conjugate/cyclosporine by weight) is 1 to 20, more preferable is 3 to 15, most preferable is 5 to 10.

Oral solution: preferable concentration of cyclosporine is 1% to 20%, more preferable is 2.5 to 10%, most preferable is 5 to 10%. The preferable ratio of conjugate to the drug (conjugate/cyclosporine) is 0,5 to 20, more preferable is 1 to 5, most preferable is 1 to 3.

IV solution; preferable concentration of cyclosporine is 0.5% to 10%, more preferable is 1 to 10%, most preferable is 1 to 5%. The preferable ratio of conjugate to the drug (conjugate/cyclosporine) is 1 to 20, more preferable is 1 to 10, most preferable is 1 to 5.

Topical solution, preferable concentration of cyclosporine is 0.05 to 1%, more preferable is 0.1 to 0.5%, most preferable is 0.1 to 0.2%. The preferable ratio of conjugate to the drug (conjugate/cyclosporine) is 1 to 20, more preferable is 3 to 15, most preferable is 5 to 10.

Oral capsule: preferable capsule content of cyclosporine is 10 mg to 200 mg, more preferable is 25 mg to 100 mg, most preferable is 50 mg to 100 mg. The preferable ratio of conjugate to the drug (conjugate/cyclosporine) is 1 to 10, more preferable is 1 to 5, most preferable is 2 to 5.

Topical cream: preferable concentration of cyclosporine is 0.05 to 2%, more preferable is 0.1 to 1%, most preferable is 0.5 to 1%. The preferable ratio of conjugate to the drug (conjugate/cyclosporine) is 1 to 20, more preferable is 3 to 15, most preferable is 5 to 10.

The invention includes a method for the treatment or prevention of protozoal infection by administering to a subject an effective inflammation treating or preventing dose of a pharmaceutical composition of cyclosporine, as well as a method for the treatment of inflammation by administering to a subject an effective inflammation treating or preventing dose of a pharmaceutical composition of cyclosporin.

For example, when treating chronic inflammations or provoking an immunosuppressive effect, it is preferred that the daily oral or injectable dose ranges from about 3 mg/kg to about 50 mg/kg in a preferred embodiment the cyclosporin is present in amounts ranging from about 1% to about 20% by weight of the pharmaceutical composition. In addition it is preferred that the cyclosporin is present in amounts ranging from about 0.05% to about 5% by weight of the pharmaceutical composition as for external uses.

In one aspect, the invention is a method of solubilizing cyclosporin. The method comprises selecting an amphipathic PEG-lipid conjugate having a melting temperature below about 25 degrees Centigrade, and adding the cyclosporin to the conjugate with mixing. The cyclosporine is mixed with the conjugate in a weight/weight ratio of conjugate to cyclosporin between about 0.5 and 20. The method may further comprising forming an aqueous solution of the conjugate either before or after mixing the cyclosporin with conjugate. The conjugate may be a DAG-PEG. The DAG-PEG may be selected from the group consisting of GDM-PEG-12, GDO-PEG-12 and GDP-PEG-12. The final cyclosporin concentration in aqueous solution may be between about 0.01 and 1 percent by weight.

In another aspect, the invention is an aqueous solution of solubilized cyclosporine comprising an amphipathic PEG-lipid conjugate having a melting temperature below about 25 degrees Centigrade, and cyclosporine at a concentration between about 0.1 and 1.0 percent by weight. The cyclosporine and the conjugate may have a weight/weight ratio of conjugate to cyclosporin between about 0.5 and 20. The conjugate may be a DAG-PEG. The DAG-PEG may be selected from the group consisting of GDM-PEG-12, GDO-PEG-12 and GDP-PEG-12. The cyclosporin concentration may be between about 0.1 and 1.0 percent by weight.

In another aspect, the invention is a pharmaceutical composition comprising an amphipathic PEG conjugate having a melting temperature below about 25 degrees Centigrade; an amphipathic PEG conjugate having a melting temperature above about 25 degrees Centigrade; cyclosporine at a weight to weight ration of between about 1 and 10 of the total amount of conjugates to cyclosporin; and a capsule coating suitable for oral ingestion. The total amount of cyclosporine may be between about 10 and 200 mg per capsule.

Other aspects of the invention includes compositions and methods for other dosage forms described herein.

The invention is illustrated in detail by the following non-limiting Examples.

### Example 1, Part 1

### Cyclosporine Ophthalmic Compositions

PEG-lipid was added to a vessel equipped with a mixer propeller. The cyclosporin drug substance was added with constant mixing. Mixing continued until the drug was visually dispersed in the lipids. Pre-dissolved excipients and sterile purified water were slowly added to the vessel with adequate mixing. Mixing continued until fully a homogenous solution was achieved. Sample formulations are described in Tables 7, 8 and 9.

TABLE 7

| Ingredient | mg/100 mL |
|---|---|
| Cyclosporin A | 5 |
| PEG-lipid | 50 |
| Sodium Hydroxide | See below |
| Hydrochloric Acid | See below |
| Sodium Chloride | 900 |
| Sterile purified water | qs 100 mL |

The PEG-lipid may be GDM-PEG₁₂, GDO-PEG₁₂, GDC-PEG₁₂ or a PEG-lipid selected from Tables 1 and 2 where the PEG chain has n polymer units ranging from 6 to 12. Combinations of PEG-lipids may also be used. Sodium hydroxide is used to prepare a 10% w/w solution in purified water. The targeted pH is in a range of 6.0 to 7.4. NaOH is used to adjust pH if necessary. The preferable cyclosporin is single form A or G or a mixture of A and G.

TABLE 8

| Ingredient | mg/100 mL |
|---|---|
| Cyclosporin A | 5 |
| PEG-lipid | 10 |
| Sodium Chloride USP | 0.9 g |
| Benzalkonium Chloride | 1:10,000 |
| Sterile purified water | qs 100 mL |

TABLE 9

| Ingredient | mg/100 mL |
|---|---|
| Cyclosporin A | 5 |
| PEG-lipid | 25 |
| Sodium Sulfite Anhydrous | 0.1 g |
| Phenylmercuric nitrate | 0.0002 |
| Disodium phosphate | 0.25 g |
| (anhydrous) | |
| Sodium chloride | 0.9 |
| Disodium EDTA | 0.1 |
| Benzalkonium chloride | 0.01 |
| Sterile Purified Water | qs ad 100 ml |

### Example 1, Part 2

### Cyclosporine Ophthalmic Compositions

PEG-lipid was added to a vessel equipped with a mixer propeller. The cyclosporine drug substance was added with constant mixing. Mixing continued until the drug was visually dispersed in the lipids. Pre-dissolved excipients and sterile purified water were slowly added to the vessel with adequate mixing. Mixing continued until fully a homogenous solution was achieved. A sample formulation is described in Table 10.

TABLE 10

| Ingredient | mg/100 mL |
|---|---|
| Cyclosporine | 50 mg |
| PEG Lipid | 500 |
| Sodium Hydroxide | See below |
| Hydrochloric Acid | See below |
| Sodium Chloride | 900 |
| Sterile purified water | qs 100 mL |

The lipid may be GDM-12, GDO-12, GDM-600, GDO-600, GDC-600, GOB-12, GMB-12, DSB-12, GMBH, GCBH or GPBH or any combination thereof. Sodium hydroxide is used to prepare a 10% w/w solution in purified water. The targeted pH is in a range of 6.0 to 7.4. NaOH is used to adjust pH if necessary.

### Example 2

### Stability of Cyclosporin Solutions

Solutions from Example 1, Part 1 were subjected to stability examinations. The solutions were stored at 5 and 25° C., respectively, after filling into amber glass vials. The assay of cyclosporin A stability samples was performed by using HPLC-UV method monitoring at UV absorbance of 220 nm, a Zorbax C8 column (300SB-C8, 4.6×100 mm, particle size 3.5 μm) was used with a mobile phase consisting of acetonitrile and 0.1% formic acid (6/4, v/v) with a flow rate of 1 mL/min. The results are summarized in Table 11.

TABLE 11

| | | Example 1 | |
|---|---|---|---|
| | | (5 mg/mL) | |
| Stability Conditions | | CyclosporinA | CyclosporinA |
| Months | °X | mg/mL | % recovery |
| Initial | | 5.03 | 100 |
| 1 | 5 | 5.05 | 100.4 |
| 1 | 25 | 5.06 | 100.6 |
| 3 | 5 | 5.04 | 100.2 |
| 3 | 25 | 5.03 | 100.0 |
| 6 | 5 | 5.01 | 99.6 |
| 6 | 25 | 4.99 | 99.2 |
| 9 | 25 | 5.00 | 99.4 |
| 12 | 25 | 5.01 | 99.6 |

### Example 3

### Comparison of Solutions from Example 1, Part 1 (“EquaSome-Cyclosporin A 0.05%” or “EC”) with Commercial Ophthalmic Emulsion for Treatment of Dry Eye Syndrome

A total of 30 rabbits with defined dry eye disease participated (5 in each treatment group). In 3 identical trials, rabbits were treated twice daily with EquaSome-Cyclosporine A (EC) 0.05%, or Commercial Ophthalmic Emulsion (COE) 0.05%, or with neither EC or RE. Adjunctive treatment with preservative-free artificial tears was undertaken four times daily in all 3 groups. Corneal fluorescein staining results, Schirmer tear test (without anesthesia) results, tear film break-up time (BUT), dry eye symptom score, and impression cytologic analysis results were obtained before treatment and at the first, second, and third months after initiation of treatment.

Both EC 0.05% and COE 0.05% treatments led to significant improvement in blurred vision, tear film BUT, and impression cytologic findings in patients with dry eye syndrome (P<0.05) compared to the control group treated with preservative-free artificial tears alone.

### Example 4

### Preparation of an Oral Capsule Containing Cyclosporin A

Charge Cyclosporin A to a suitable vessel equipped with a mixer propeller. Add liquid PEG-lipid with constant mixing. Continue mixing until fully dispersed. Slowly add a solid PEG-lipid to the vessel with constant mixing. Mix the lipid with slow agitation until all solids are visually dispersed in the solution. Keep warm and transfer the mixture to the filling steps.

Set-up the appropriate filling equipment (e.g. Bosch's GKF 1400L) with the required fill volume. Fill the liquid into the shells of capsules. Maintain agitation of the batch until the liquid level falls below the bottom agitator blade. Fill the remainder of the batch within one hour after agitation is stopped. Encapsulate cyclosporine final blend into opaque hard gelatin capsule shells at a target fill weight. Transfer the finished capsules into a suitable closed cool chamber container 0-4° C. over night to let the capsule content become solidified. A sample formula is listed in Table 12.

TABLE 12

| Ingredient | mg/cap |
|---|---|
| Cyclosporin A (or G) | 100.0 |
| Preservative | 50 |
| PEG-lipid (liquid)¹ | 200.0 |
| PEG-lipid (solid)² | 200.0 |

¹1,2-dimystryl-rac-glycerol-3-mPEG-12 or see Table 1 and 2, where the n = 6 to 12
²1,2-diseraoyl-rac-glycerol-3-mPEG-23 or or see Table 1 and 2, where the n = 18 to 23

The preferable cyclosporin is single form A or G or a mixture of A and G. The preferable cyclosporin content is 50 mg to 200 mg per capsule and the ratio of PEG-lipid to the drug is 2 to 5.

### Example 5

### Cyclosporine Ophthalmic Compositions

PEG-lipid was added to a vessel equipped with a mixer propeller. The cyclosporin drug substance was added with constant mixing. Mixing continued until the drug was visually dispersed in the lipids. Pre-dissolved excipients and sterile purified water were slowly added to the vessel with adequate mixing. Mixing continued until fully a homogenous solution was achieved. The pH value of the solution was determined and adjusted to be 6.6 to 8 at 25° C. The solution was then filtered through a sterile filter with a pore size of 0.2 μm and filled into appropriate containers under aseptic conditions. A sample formulation is described in Table 13.

TABLE 13

| Ingredient | %/100 mL | | | |
|---|---|---|---|---|
| Cyclosporin | 1 | 0.5 | 0.1 | 0.05 |
| A | | | | |
| PEG-lipid | 10 | 5 | 1 | 0.5 |
| Sodium | See below | See below | See below | See below |
| Hydroxide | | | | |
| Hydrochloric | See below | See below | See below | See below |
| Acid | | | | |
| Sodium | 0.9 | 0.9 | 0.9 | 0.9 |
| Chloride | | | | |
| Benzyl | 1 | 1 | 1 | 1 |
| alcohol | | | | |
| Sterile purified | qs 100 mL | qs 100 mL | qs 100 mL | qs 100 mL |
| water | | | | |

The PEG-lipid may be GDM-PEG₁₂, GDO-PEG₁₂, GDC-PEG₁₂ or a PEG-lipid selected from Tables 1 and 2 where the n is ranging from 6 to 12 or any combination thereof. Sodium hydroxide is used to prepare a 10% w/w solution in purified water. The targeted pH is in a range of 6.5 to 7.5. NaOH is used to adjust pH if necessary.

The preferable cyclosporin is single form A or G or a mixture of A and G.

Following a similar fashion in the Example 1, an ophthalmic gel can also be made by adding Carbopol (i.e., Carbopol 980 NF®) as described in Table 14.

TABLE 14

| Ingredient | %/100 mL | | | |
|---|---|---|---|---|
| Cyclosporin | 1 | 0.5 | 0.1 | 0.05 |
| A | | | | |
| PEG-lipid | 10 | 5 | 1 | 0.5 |
| Carbomer | 2 | 2 | 2 | 2 |
| 980 | | | | |
| Sodium | See below | See below | See below | See below |
| Hydroxide | | | | |
| Hydrochloric | See below | See below | See below | See below |
| Acid | | | | |
| Sodium | 0.9 | 0.9 | 0.9 | 0.9 |
| Chloride | | | | |
| Sterile purified | qs 100 mL | qs 100 mL | qs 100 mL | qs 100 mL |
| water | | | | |

### Example 6

### Preparation of an Oral Solution Containing Cyclosporin

Charge cyclosporin to a suitable vessel equipped with a mixer propeller. Add lactic acid with gentle mixing to levigate the drug powder. Add PEG-lipid with constant mixing. Continue mixing until fully dispersed.

Charge 30-35% of the final batch volume of purified water to a suitable container and add the sodium benzoate and sodium citrate to the container and mix with a propeller mixer for approximately five minutes. Slowly add the xanthan gum to the container with constant mixing. Mix until the xanthan gum is visually dispersed in the solution.

Slowly add the premix in to the vessel with adequate mixing. Use purified water to rinse any remaining xanthan gum from the agitator shaft and sides of the container, and charge the rinse to the vessel with adequate mixing. A sample formulation is listed in Table 15.

TABLE 15

| Ingredient | mg/mL |
|---|---|
| Cyclosporin A (G) | 50 |
| PEG-lipid | 200 |
| Sodium Citrate, Dihydrate | 2 |
| Lactic acid | 5 |
| Xanthan Gum | 3.0 |
| Sodium Benzoate | 2.0 |
| Liquid Glucose | 350.0 |
| Artificial Flavor | 5.0 |
| (i.e., Virginia Dare # 13174) | |
| Purified Water | qs ad 1 mL |

The PEG-lipid may be GDM-PEG₁₂, GDO-PEG₁₂, GDC-PEG₁₂ or a PEG-lipid selected from Tables 1 and 2 where the n is ranging from 6 to 12 or any combination thereof. Organic acid may be lactic acid or pyruvic acid or glycolic acid. Sodium hydroxide is used to adjust pH if necessary. The targeted pH range was between 3.5 and 7.0.

The preferable cyclosporin is single form A or G or a mixture of A and G.

### Example 7

### Preparation of a Topical Cream Containing Cyclosporin

PEG lipid was added to a stainless steel vessel equipped with propeller type mixing blades. The drug substance was added with constant mixing. Mixing continued until the drug was visually dispersed in the lipids at a temperature to 60°-65° C. Organic acid, Cholesterol and glycerin were added with mixing. Ethanol and ethyoxydiglycol were added with mixing. Finally Carbopol ETD 2020, purified water and triethylamine were added with mixing. Mixing continued until fully a homogenous cream was achieved. The formulation is described in Table 16.

TABLE 16

| Ingredient | % |
|---|---|
| Drug Substance (Active) | 0.1 |
| PEG-lipid | 5.0 |
| Carbopol ETD 2020 | 0.5 |
| Ethyoxydiglycol | 1.0 |
| Ethanol | 5.0 |
| Glycerin | 1.0 |
| Cholesterol | 0.4 |
| Triethylamine | 0.20 |
| Organic acid | 10 |
| Sodium hydroxide | See below |
| Purified water | qs 100 |

The PEG-lipid may be GDM-PEG₁₂, GDO-PEG₁₂, GDC-PEG₁₂ or a PEG-lipid selected from Tables 1 and 2 where the n is ranging from 6 to 12 or any combination thereof. Organic acid may be lactic acid or pyruvic acid or glycolic acid. Sodium hydroxide is used to adjust pH if necessary. The targeted pH range was between 3.5 and 7.0.

The preferable cyclosporin is single form A or G or a mixture of A and G.

### Example 8

### Preparation Topical Solution Containing Cyclosporin

The topical solution was prepared as above, a sample formulation is described in Table 17.

TABLE 17

| Ingredient | % |
|---|---|
| Drug Substance (Active) | 0.5 |
| PEG Lipid | 5.0 |
| α-Tocopherol | 0.5 |
| Organic acid | 10.0 |
| Ethanol | 5.0 |
| Sodium Benzoate | 0.2 |
| Sodium Hydroxide | See Below |
| Purified Water | qs 100 |

The PEG-lipid may be GDM-PEG₁₂, GDO-PEG₁₂, GDC-PEG₁₂ or a PEG-lipid selected from Tables 1 and 2 where the n is ranging from 6 to 12 or any combination thereof. Organic acid may be lactic acid or pyruvic acid or glycolic acid. Sodium hydroxide is used to adjust pH if necessary. The targeted pH range was between 3.5 and 7.0.

The preferable cyclosporin is single form A or G or a mixture of A and G.

### Example 9

### Preparation Injectable Solution Containing Cyclosporin

Charge lactic acid and cyclosporin into a stainless steel vessel while maintaining mixing at 250-400 RPM, the mixing rate can be adjusted to assist in wetting Charge and disperse the required amount of PEG-lipid into the vessel with agitation at a speed of 300 RPM. Agitation speed can be adjusted to assist in dispersing PEG-lipid, but foaming must be avoided. Maintain an overlay of sterile-filtered nitrogen on the vessel after the PEG-lipid has dispersed. Monitor the mixing continuously until no large drug substance powder agglomerates are observed. Stop mixing and homogenize the lipid solution at 850-900 RPM until a homogenous lipid phase is observed. Avoid foaming of premix. Maintain an overlay of sterile-filtered nitrogen on the vessel. Other compositions are described in Table 18 are premixed in purified water then add into the vessel with constant mixing. Fill the product in a sterile-filtered nitrogen environment into washed and sterilized glass vials. Purge each vial with sterile-filtered nitrogen prior to filling and overlay with sterile-filtered nitrogen after filling.

TABLE 18

| | Amount |
|---|---|
| Ingredient | (mg/mL) |
| Drug (active) | 5 |
| PEG-lipid | 30 |
| Organic acid | 10 |
| Sodium Phosphate, Monobasic | 0.040 |
| Sodium Phosphate, Dibasic, | 1.378 |
| Sodium Hydroxide | For pH Adjustment |
| Phosphoric Acid | For pH Adjustment |
| Water for Injection | 1.0 mL |

The PEG-lipid may be GDM-PEG₁₂, GDO-PEG₁₂, GDC-PEG₁₂ or a PEG-lipid selected from Tables 1 and 2 where the n is ranging from 6 to 12 or any combination thereof. Organic acid may be lactic acid or pyruvic acid or glycolic acid. Sodium hydroxide is used to adjust pH if necessary. The targeted pH range was between 6.0 and 7.5.

The preferable cyclosporin is single form A or G or a mixture of A and G.

### Example 10

### Stability of Cyclosporin Solutions

Examples 5, 6 and 9 were subjected to stability examinations. The solutions were stored at 5 and 25° C., respectively, after filling into amber glass vials.

The assay of cyclosporin A stability samples was performed by using HPLC-UV method monitoring at UV absorbance of 220 nm, a Zorbax C8 column (300SB-C8, 4.6×100 mm, particle size 3.5 μm) was used with a mobile phase consisting of acetonitrile and 0.1% formic acid (6/4, v/v) with a flow rate of 1 mL/min. The results are summarized in Table 19.

TABLE 19
Package: 20-mL amber glass bottle with white polypropylene cap

| | | Example 2 | | Example 3 | | Example 6 | |
|---|---|---|---|---|---|---|---|
| Stability | | (5 mg/mL) | | (50 mg/mL) | | (5 mg/mL) | |
| Conditions | | CyclosporinA | CyclosporinA | CyclosporinA | CyclosporinA | CyclosporinA | CyclosporinA % |
| Months | °X | mg/mL | % recovery | mg/mL | % recovery | mg/mL | recovery |
| Initial | | 1.03 | 100.0 | 51.3 | 100.0 | 4.98 | 100.0 |
| 1 | 5 | 1.05 | 101.9 | 52.1 | 101.6 | 5.03 | 101.0 |
| 1 | 25 | 1.06 | 102.9 | 52.4 | 102.1 | 5.08 | 102.0 |
| 3 | 5 | 1.04 | 101.0 | 51.1 | 99.6 | 5.03 | 101.0 |
| 3 | 25 | 1.03 | 100.0 | 50.8 | 99.0 | 5.01 | 100.6 |
| 6 | 5 | 1.01 | 98.1 | 51.6 | 100.6 | 5.03 | 101.0 |
| 6 | 25 | 0.99 | 96.2 | 50.8 | 99.1 | 4.94 | 99.1 |
| 9 | 25 | 1.00 | 97.1 | 51.3 | 100.0 | 4.98 | 100.0 |
| 12 | 25 | 1.01 | 98.1 | 50.8 | 99.0 | 5.01 | 100.6 |

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

We claim:

1. A method of solubilizing cyclosporin, the method comprising:
selecting an amphipathic PEG-lipid conjugate having a melting temperature below about 25 degrees Centigrade; and
adding the cyclosporin to the conjugate with mixing.

2. The method of claim 1, cyclosporine is mixed with the conjugate in a weight/weight ratio of conjugate to cyclosporin between about 0.5 and 20.

3. The method of claim 1, further comprising forming an aqueous solution of the conjugate after said adding.

4. The method of claim 1, where the conjugate is a DAG-PEG.

5. The method of claim 4, where the DAG-PEG is selected from the group consisting of GDM-PEG-12, GDO-PEG-12 and GDP-PEG-12.

6. The method of claim 3, where the final cyclosporin concentration is between about 0.01 and 1 percent by weight.

7. An aqueous solution of solubilized cyclosporine comprising
an amphipathic PEG-lipid conjugate having a melting temperature below about 25 degrees Centigrade; and
cyclosporine at a concentration between about 0.1 and 1.0 percent by weight.

8. The solution of claim 7, where the cyclosporine and the conjugate have a weight/weight ratio of conjugate to cyclosporin between about 0.5 and 20.

9. The solution of claim 7, where the conjugate is a DAG-PEG.

10. The solution of claim 9, where the DAG-PEG is selected from the group consisting of GDM-PEG-12, GDO-PEG-12 and GDP-PEG-12.

11. The solution of claim 7, where the cyclosporin concentration is between about 0.1 and 1.0 percent by weight.

12. A pharmaceutical composition comprising:
a first amphipathic PEG conjugate, said first conjugate having a melting temperature below about 25 degrees Centigrade;
a second amphipathic PEG conjugate having a melting temperature above about 25 degrees Centigrade;
cyclosporine at a weight to weight ration of between about 1 and 10 of the total amount of conjugates to cyclosporin; and
a capsule coating suitable for oral ingestion.

13. The composition of claim 12, where the total amount of cyclosporine is between about 10 and 200 mg.
